(19) United States
(12) Patent Application Publication
(10) Pub. No.: US 2022/0162700 A1
(43) Pub. Date: May 26, 2022

(54) NOVEL METHOD FOR MONITORING ENDOMETRIOSIS AND ASSOCIATED DISORDERS

(71) Applicants: Li Chen, Columbus, OH (US); Yu Chou, Columbus, OH (US); RESEARCH INSTITUTE AT NATIONWIDE CHILDREN'S HOSPITAL, Columbus, OH (US)

(72) Inventors: Li Chen, Columbus, OH (US); Yu Zhou, Columbus, OH (US); David Brigstock, Columbus, OH (US); Gail Besner, Columbus, OH (US)

(73) Assignee: RESEARCH INSTITUTE AT NATIONWIDE CHILDREN'S HOSPITAL, Columbus, OH (US)

(21) Appl. No.: 17/367,201

(22) Filed: Jul. 2, 2021

Related U.S. Application Data
(63) Continuation of application No. PCT/US2020/012802, filed on Jan. 8, 2020.
(60) Provisional application No. 62/790,337, filed on Jan. 9, 2019.

(51) Int. Cl. C12Q 1/6883; C12N 15/113; C12N 15/10

(52) U.S. Cl. CPC C12Q 1/6883; C12N 15/113; C12N 15/10; C12N 2320/30; C12Q 2600/106; C12Q 2600/178; C12N 2310/141; C12Q 2600/158

(57) ABSTRACT

The present invention provides novel methods and kits for determining whether a subject is suffering from endometriosis or an associated disorder. It also provides methods and kits for determining whether a subject suffering from endometriosis or an associated disorder is more or less likely to be responsive to a therapy to treat endometriosis or an associated disorder.

### CROSS-REFERENCE TO RELATED APPLICATION

This application is a continuation application filed under 35 U.S.C. § 111(a) claiming the benefit under 35 U.S.C. § 120 and 365(c) of International Patent Application No. PCT/US2020/012802, filed Jan. 8, 2020, which claims priority under 35 U.S.C. § 119(e) to Provisional Application, U.S. Ser. No.: 62/790,337, filed Jan. 9, 2019, the contents of each of which are hereby incorporated by reference in their entireties into the present disclosure.

### TECHNICAL FIELD

The present disclosure relates to a painless and simple new method for whether a subject is suffering from endometriosis or an associated disorder

### BACKGROUND

Endometriosis is a chronic gynecological disorder characterized by the development of endometrial tissue outside of the uterus. Women who suffer from endometriosis may also suffer from chronic paid and infertility. Although the exact cause of endometriosis is unknown, it is believed to be related to fibrosis.

### SUMMARY

Fibrosis is an important pathological feature of endometriosis (EMs), which is a major factor in female pelvic pain and infertility. It is disclosed that the ectopic endometrium of endometriosis (EMs) patients have significantly decreased expression of miR-214, and overactive CCN2, which promotes ectopic endometrium fibrosis and leads to infertility. After treated with miR-214 or miR-214 mimics, the production of CCN2. Collagen I or αSMA protein can be reduced in cell and animal experiments. It is also disclosed that EMs patients have significantly decreased miR-214 level in the serum as compared to normal control. Given miR-214 is mainly packaged and cargoed by exosome and that a kind of nanometer sized extracellular vesicles shuttled between cells and released into circulation, provided herein are exosomal miR-214 that directly target CTGF to inhibit the fibrosis in endometriosis. Also disclosed is the delivery of exosomes with high copies of miR-214 to EMs in subjects to inhibit the fibrosis of ectopic endometrium and improve the prognosis of EMs patients. In addition, circulating exosome carried miR-214 can serve as a non-invasive biomarker for early detection of EMs.

Provided herein are methods of determining whether a subject is suffering from endometriosis or an associated disorder, comprising, consisting essentially of, or yet consisting of determining the expression level of miR-214 in a sample enriched from a subject, wherein reduced expression of the miR-214 as compared to a control is indicative of the subject suffering from endometriosis or an associated disorder. Normal or reduced expression is indicative that the subject is not likely suffering from endometriosis or an associated disorder.

Also provided are methods of determining whether a subject suffering from endometriosis or an associated disorder is more or less likely to be responsive to a therapy to treat endometriosis or an associated disorder, comprising, or consisting essentially of, or yet consisting determining the expression level of miR-214 in a sample enriched from the subject, wherein reduced expression of the miR-214 as compared to a control indicates the subject is more likely to be responsive to therapy and/or wherein increased expression of the miR-214 as compared to a control indicates the subject is less likely to be responsive to the therapy.

### DETAILED DESCRIPTION

Throughout this disclosure, various publications, patents and published patent specifications are referenced by an identifying citation. The disclosures of these publications, patents and published patent specifications are hereby incorporated by reference into the present disclosure to more fully describe the state of the art to which this disclosure pertains.

The practice of the present disclosure employs, unless otherwise indicated, techniques of molecular biology (including recombinant techniques), microbiology, cell biology, biochemistry and immunology, which are within the skill of the art. Such techniques are explained fully in the literature for example in the following publications. See, e.g., Sambrook and Russell eds. MOLECULAR CLONING: A LABORATORY MANUAL, 3^{rd} edition (2001); the series CURRENT PROTOCOLS IN MOLECULAR BIOLOGY (F. M. Ausubel et al. eds. (2007)); the series METHODS IN ENZYMOLOGY (Academic Press, Inc., N.Y.); PCR 1: A PRACTICAL APPROACH (M. MacPherson et al. IRL Press at Oxford University Press (1991)); PCR 2: A PRACTICAL APPROACH (M. J. MacPherson, B. D. Hames and G. R. Taylor eds. (1995)); ANTIBODIES, A LABORATORY MANUAL (Harlow and Lane eds. (1999)); CULTURE OF ANIMAL CELLS: A MANUAL OF BASIC TECHNIQUE (R.I. Freshney 5^{th} edition (2005)); OLIGONUCLEOTIDE SYNTHESIS (M. J. Gait ed. (1984)); Mullis et al. U.S. Pat. No. 4,683,195; NUCLEIC ACID HYBRIDIZATION (B. D. Hames & S. J. Higgins eds. (1984)); NUCLEIC ACID HYBRIDIZATION (M. L. M. Anderson (1999)); TRANSCRIPTION AND TRANSLATION (B. D. Hames & S. J. Higgins eds. (1984)); IMMOBILIZED CELLS AND ENZYMES (IRL Press (1986)); B. Perbal, A PRACTICAL GUIDE TO MOLECULAR CLONING (1984); GENE TRANSFER VECTORS FOR MAMMALIAN CELLS (J. H. Miller and M. P. Calos eds. (1987) Cold Spring Harbor Laboratory); GENE TRANSFER AND EXPRESSION IN MAMMALIAN CELLS (S. C. Makrides ed. (2003)) IMMUNOCHEMICAL METHODS IN CELL AND MOLECULAR BIOLOGY (Mayer and Walker, eds., Academic Press, London (1987)); WEIR'S HANDBOOK OF EXPERIMENTAL IMMUNOLOGY (L. A. Herzenberg et al. eds (1996)).

Definitions

As used herein, certain terms may have the following defined meanings. As used in the specification and claims, the singular form “a,” “an” and “the” include singular and plural references unless the context clearly dictates otherwise. For example, the term “a cell” includes a single cell as well as a plurality of cells, including mixtures thereof.

As used herein, the term “comprising” is intended to mean that the compositions and methods include the recited elements, but not excluding others. “Consisting essentially of” when used to define compositions and methods, shall mean excluding other elements of any essential significance to the composition or method. “Consisting of” shall mean excluding more than trace elements of other ingredients for claimed compositions and substantial method steps. Embodiments defined by each of these transition terms are within the scope of this disclosure. Accordingly, it is intended that the methods and compositions can include additional steps and components (comprising) or alternatively including steps and compositions of no significance (consisting essentially of) or alternatively, intending only the stated method steps or compositions (consisting of).

All numerical designations, e.g., pH, temperature, time, concentration, and molecular weight, including ranges, are approximations which are varied (+) or (−) by increments of 0.1. It is to be understood, although not always explicitly stated that all numerical designations are preceded by the term “about”. The term “about” also includes the exact value “X” in addition to minor increments of “X” such as “X+0.1” or “X−0.1.” It also is to be understood, although not always explicitly stated, that the reagents described herein are merely exemplary and that equivalents of such are known in the art.

As used herein, the term “endometriosis” is understood to be a chronic gynecological disorder characterized by the development of endometrial tissue outside of the uterus. An “associated disorder” is a disease or condition related to this development of endometrial tissue outside of the uterus, e.g., fibrosis, chronic pelvic pain, infertility, excess fibrosis, scarring, altered tissue function and resistance to hormonal suppressive therapy.

As used herein, the term “exosome” intends a membrane body having an average diameter of from about 10 nm to about 2,000 nm. The term includes microvesicles and exosomes. Microvesicles are also known as circulating microvesicles or microparticles and are fragments of plasma membrane ranging from 100 nm to 1000 nm in approximate diameter shed from almost all cell types. For the purpose of this disclosure and unless specifically noted, the term exosome also includes smaller intracellularly generated extracellular vesicles formed by inward budding of the limiting membranes of multivesicular bodies (MVB) which, upon fusion with the plasma membrane, result in their secretion and deposition into body fluids (e.g., blood, urine). Exosomes contain a complex mixture of microRNAs (miRs), mRNAs and proteins that reflect the transcriptional and translational status of the producer cell. Exosomes are from about 10 to about 250 nm, or alternatively from about 10 to about 200 nm, or alternatively from about 10 to about 175 nm, or alternatively from about 25 to 175 nm, or alternatively from about 40 to about 250 nm, or alternatively from about 40 to about 200 nm, or alternatively from about 50 to 250 nm, or alternatively from about 50 to 200 nm, or alternatively from about 50-150 nm in average diameter. The exosome membranous vesicles arise by inward budding from the limiting membranes of MVB. Upon fusion of MVBs with the plasma membrane, exosomes are liberated from the cells, traverse intercellular spaces, and may be taken up by neighboring cells (Johnstone, R. M. (2006) Blood Cells Mol. Dis. 36(2):315-321; Thery, C. (2011) F1000 Biol. Rep. 3:15; Thery, C. et al. (2002) Nat. Rev. Immunol. 2(8):569-579). Exosomes contain a complex mixture of miRs, mRNAs and proteins and can be enriched from a variety of body fluids as described herein and known in the art.

The term “identify” or “identifying” is to associate or affiliate a patient closely to a group or population of patients who likely experience the same or a similar clinical response to treatment.

The terms “protein,” “polypeptide” and “peptide” are used interchangeably herein when referring to a gene product.

The term “marker” refers to a clinical or sub-clinical expression of a gene or miRNA of interest.

“Expression” as applied to a gene, refers to the differential production of the miR or mRNA transcribed from the gene or the protein product encoded by the gene. A differentially expressed gene may be over expressed (high expression) or under expressed (low expression) as compared to the expression level of a normal or control cell, a given patient population or with an internal control gene (housekeeping gene). In one aspect, it refers to a differential that is about 1.5 times, or alternatively, about 2.0 times, alternatively, about 2.0 times, alternatively, about 3.0 times, or alternatively, about 5 times, or alternatively, about 10 times, alternatively about 50 times, or yet further alternatively more than about 100 times higher or lower than the expression level detected in a control sample.

In one aspect of the disclosure, a “predetermined threshold level” or “threshold value” is used to categorize expression as high or low. As a non-limiting example of the disclosure, the threshold level of the miR of the exosome is a level of miR expression found in subjects that have been diagnosed with a fibrotic or hepatic disease or an associate disorder. Alternatively, or in addition, the predetermined threshold level is the measured miRNA expression level for that individual subject prior to a subsequent measurement, e.g., prior to therapy or prior to an additional dose of the therapy.

In one aspect of the disclosure, miR expression can be provided as a ratio above the threshold level and therefore can be categorized as high expression or up-regulated, whereas a ratio below the threshold level is categorized as down-regulated or low expression.

In another aspect, “expression” level is determined by measuring the expression level of a miR of interest for a given patient population, determining the median expression level of that miR for the population, and comparing the expression level of the same miR for a single patient to the median expression level for the given patient population. For example, if the expression level of a miR of interest for the single patient is determined to be above the median expression level of the patient population, that patient is determined to have high expression (up-regulated) of the miR of interest. Alternatively, if the expression level of a miR of interest for the single patient is determined to be below the median expression level (down-regulated) of the patient population, that patient is determined to have low expression of the miR of interest.

A “internal control” or “housekeeping” gene refers to any constitutively or globally expressed gene whose presence enables an assessment of the expression level of a gene or genes of interest. Such an assessment comprises a determination of the overall constitutive level of gene transcription and a control for variation in sampling error. Examples of such genes include, but are not limited to, RNU6-2, cel-miR-39, SNORD61, SNORD68, SNORD72, SNORD95, SNORD96A, GADPH and/or β-actin.

“Cells,” “host cells” or “recombinant host cells” are terms used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

The phrase “amplification of polynucleotides” includes methods such as PCR, ligation amplification (or ligase chain reaction, LCR) and amplification methods. These methods are known and widely practiced in the art. See, e.g., U.S. Pat. Nos. 4,683,195 and 4,683,202 and Innis et al., 1990 (for PCR); and Wu, D. Y. et al. (1989) Genomics 4:560-569 (for LCR). In general, the PCR procedure describes a method of gene amplification which is comprised of (i) sequence-specific hybridization of primers to specific genes within a DNA sample (or library), (ii) subsequent amplification involving multiple rounds of annealing, elongation, and denaturation using a DNA polymerase, and (iii) screening the PCR products for a band of the correct size. The primers used are oligonucleotides of sufficient length and appropriate sequence to provide initiation of polymerization, i.e., each primer is specifically designed to be complementary to each strand of the genomic locus to be amplified.

Reagents and hardware for conducting PCR are commercially available. Primers useful to amplify sequences from a particular gene region are preferably complementary to, and hybridize specifically to sequences in the target region or in its flanking regions. Nucleic acid sequences generated by amplification may be sequenced directly. Alternatively, the amplified sequence(s) may be cloned prior to sequence analysis. A method for the direct cloning and sequence analysis of enzymatically amplified genomic segments is known in the art.

The term “encode” as it is applied to polynucleotides refers to a polynucleotide which is said to “encode” a polypeptide if, in its native state or when manipulated by methods well known to those skilled in the art, it can be transcribed from its gene and/or translated from its mRNA to produce the polypeptide and/or a fragment thereof. The antisense strand is the complement of such a nucleic acid, and the encoding sequence can be deduced therefrom.

“Homology” or “identity” or “similarity” refers to sequence similarity between two peptides or between two nucleic acid molecules. Homology can be determined by comparing a position in each sequence which may be aligned for purposes of comparison. When a position in the compared sequence is occupied by the same base or amino acid, then the molecules are homologous at that position. A degree of homology between sequences is a function of the number of matching or homologous positions shared by the sequences. An “unrelated” or “non-homologous” sequence shares less than 40% identity, though preferably less than 25% identity, with one of the sequences of the present disclosure.

A polynucleotide or polynucleotide region (or a polypeptide or polypeptide region) has a certain percentage (for example, 60%, 65%, 70%, 75%, 80%, 85%, 90%, 95%, 98% or 99%) of “sequence identity” to another sequence means that, when aligned, that percentage of bases (or amino acids) are the same in comparing the two sequences. This alignment and the percent homology or sequence identity can be determined using software programs known in the art, for example those described in Ausubel et al. eds. (2007) Current Protocols in Molecular Biology. Preferably, default parameters are used for alignment. One alignment program is BLAST, using default parameters. In particular, programs are BLASTN and BLASTP, using the following default parameters: Genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+SwissProtein+SPupdate+PIR. Details of these programs can be found at the following Internet address: http://www.ncbi.nlm.nih.gov/blast/Blast.cgi, last accessed on Nov. 26, 2007. Equivalent polynucleotides are those having the specified percent homology and/or encoding a polypeptide having the same or similar biological activity.

High stringency hybridization conditions is generally performed at about 60° C. in about 1×SSC. Substantially homologous and equivalent polynucleotide and/or polypeptides intend those having at least 80% homology, or alternatively at least 85% homology, or alternatively at least 90% homology, or alternatively, at least 95% homology or alternatively, at least 98% homology to those described above, each as determined using methods known to those skilled in the art and identified herein, when run under default parameters. They may possess at least 60%, or alternatively, at least 65%, or alternatively, at least 70%, or alternatively, at least 75%, or alternatively, at least 80%, or alternatively at least 85%, or alternatively at least 90%, or alternatively at least 95% or alternatively at least 98%, identical primary amino acid sequence to the reference polypeptide or polynucleotide when compared using sequence identity methods run under default conditions. In one specific aspect, they may possess at least 60%, or alternatively, at least 65%, or alternatively, at least 70%, or alternatively, at least 75%, or alternatively, at least 80%, or alternatively at least 85%, or alternatively at least 90%, or alternatively at least 95% or alternatively at least 98%, identical primary amino acid or polynucleotide sequence to the reference polypeptide when compared using sequence identity methods run under default conditions.

The term “interact” as used herein is meant to include detectable interactions between molecules, such as can be detected using, for example, a hybridization assay. The term interact is also meant to include “binding” interactions between molecules. Interactions may be, for example, protein-protein, protein-nucleic acid, protein-small molecule or small molecule-nucleic acid in nature.

A composition that is “enriched” for exosomes refers to a composition in which the concentration of exosomes is increased relative to the volume or to other non-exosome components. Exosome enrichment of a composition can be accomplished by separating (colloquially referred to as purifying or isolating) exosomes from other non-exosome entities. In other words, the concentration of exosomes in the composition to which the separation technique was applied will be increased relative to the other non-exosome components.

The term “isolated” as used herein refers to molecules or biological or cellular materials being substantially free from other materials. In one aspect, the term “isolated” refers to nucleic acid, such as DNA or RNA, or protein or polypeptide, or cell or cellular organelle, or tissue or organ, separated from other DNAs or RNAs, or proteins or polypeptides, or cells or cellular organelles, or tissues or organs, respectively, that are present in the natural source. The term “isolated” also refers to a nucleic acid or peptide that is substantially free of cellular material, viral material, or culture medium when produced by recombinant DNA techniques, or chemical precursors or other chemicals when chemically synthesized. Moreover, an “isolated nucleic acid” is meant to include nucleic acid fragments which are not naturally occurring as fragments and would not be found in the natural state. The term “isolated” is also used herein to refer to polypeptides which are isolated from other cellular proteins and is meant to encompass both purified and recombinant polypeptides. The term “isolated” is also used herein to refer to cells or tissues that are isolated from other cells or tissues and is meant to encompass both cultured and engineered cells or tissues.

“Expression” as applied to a gene, refers to the production of the miR or mRNA transcribed from the gene, or the protein product encoded by the mRNA. The expression level of a gene may be determined by measuring the amount of miR or mRNA or protein in a cell or tissue sample. In one aspect, the expression level of a gene is represented by a relative level as compared to a housekeeping gene as an internal control. In another aspect, the expression level of a gene from one sample may be directly compared to the expression level of that gene from a different sample using an internal control to remove the sampling error.

“Differential expression,” “overexpression” or “underexpression” refers to increased or decreased expression, or alternatively a differential expression, of a miR in a test sample as compared to the expression level of that miR in the control sample. In one aspect, the test sample is a diseased cell, and the control sample is a normal cell. In another aspect, the test sample is an experimentally manipulated or biologically altered cell, and the control sample is the cell prior to the experimental manipulation or biological alteration. In yet another aspect, the test sample is a sample from a patient, and the control sample is a similar sample from a healthy individual. In a yet further aspect, the test sample is a sample from a patient and the control sample is a similar sample from patient not having the desired clinical outcome. In one aspect, the differential expression is about 1.5 times, or alternatively, about 2.0 times, or alternatively, about 2.0 times, or alternatively, about 3.0 times, or alternatively, about 5 times, or alternatively, about 10 times, or alternatively about 50 times, or yet further alternatively more than about 100 times higher or lower than the expression level detected in the control sample. Alternatively, the miR is referred to as “over expressed” or “under expressed”. Alternatively, the miR may also be referred to as “up regulated” or “down regulated.”

A “predetermined value” for a miR as used herein, is so chosen that a patient with an expression level of that miR higher than the predetermined value is likely to experience a more or less desirable clinical outcome than patients with expression levels of the same miR lower than the predetermined value, or vice-versa. Expression levels of miR, such as those disclosed in the present disclosure, are associated with clinical outcomes. One of skill in the art can determine a predetermined value for a miR by comparing expression levels of a miR in patients with more desirable clinical outcomes to those with less desirable clinical outcomes. In one aspect, a predetermined value is a miR expression value that best separates patients into a group with more desirable clinical outcomes and a group with less desirable clinical outcomes. Such a miR expression value can be mathematically or statistically determined with methods well known in the art.

Alternatively, a miR expression that is higher than the predetermined value is simply referred to as a “high expression”, or a miR expression that is lower than the predetermined value is simply referred to as a “low expression.”

Briefly and for the purpose of illustration only, one of skill in the art can determine a predetermined value by comparing expression values of a miR in patients with more desirable clinical parameters to those with less desirable clinical parameters. In one aspect, a predetermined value is a miR expression value that best separates patients into a group with more desirable clinical parameter and a group with less desirable clinical parameter. Such a miR expression value can be mathematically or statistically determined with methods well known in the art.

As used herein, the term “nucleic acid” refers to polynucleotides such as deoxyribonucleic acid (DNA), and, where appropriate, ribonucleic acid (RNA). The term should also be understood to include, as equivalents, derivatives, variants and analogs of either RNA or DNA made from nucleotide analogs, and, as applicable to the embodiment being described, single (sense or antisense) and double-stranded polynucleotides. Deoxyribonucleotides include deoxyadenosine, deoxycytidine, deoxyguanosine, and deoxythymidine. For purposes of clarity, when referring herein to a nucleotide of a nucleic acid, which can be DNA or an RNA, the terms “adenosine,” “cytidine,” “guanosine,” and “thymidine” are used. It is understood that if the nucleic acid is RNA, a nucleotide having an uracil base is uridine.

The terms “oligonucleotide” or “polynucleotide,” or “portion,” or “segment” thereof refer to a stretch of polynucleotide residues which is long enough to use in PCR or various hybridization procedures to identify or amplify identical or related parts of miR or mRNA or DNA molecules. The polynucleotide compositions of this disclosure include miR, RNA, cDNA, genomic DNA, synthetic forms, and mixed polymers, both sense and antisense strands, and may be chemically or biochemically modified or may contain non-natural or derivatized nucleotide bases, as will be readily appreciated by those skilled in the art. Such modifications include, for example, labels, methylation, substitution of one or more of the naturally occurring nucleotides with an analog, internucleotide modifications such as uncharged linkages (e.g., methyl phosphonates, phosphotriesters, phosphoamidates, carbamates, etc.), charged linkages (e.g., phosphorothioates, phosphorodithioates, etc.), pendent moieties (e.g., polypeptides), intercalators (e.g., acridine, psoralen, etc.), chelators, alkylators, and modified linkages (e.g., alpha anomeric nucleic acids, etc.). Also included are synthetic molecules that mimic polynucleotides in their ability to bind to a designated sequence via hydrogen bonding and other chemical interactions. Such molecules are known in the art and include, for example, those in which peptide linkages substitute for phosphate linkages in the backbone of the molecule.

microRNAs, miRNAs, or miRs are single-stranded RNA molecules of 19-25 nucleotides in length, which regulate gene expression. miRNAs are encoded by genes from whose DNA they are transcribed but miRNAs are not translated into protein (non-coding RNA); instead each primary transcript (a pri-miRNA) is processed into a short stem-loop structure called a pre-miRNA and finally into a functional miRNA. Mature miRNA molecules are partially complementary to one or more messenger RNA (mRNA) molecules, and their main function is to down-regulate gene expression.

The sequence for human miR-214 is known in the art and disclosed for example at mirbase.org/cgi-bin/mirna_entry.pl?acc=MI0000290 (accessed on Jan. 8, 2019). The miR-214 family is disclosed at http://www.mirbase.org/summary.shtml?fam=MIPF000062 (accessed on Jan. 8, 2019).

Human miR-214 reproduced from mirbase.org/cgi-bin/mima_entry.pl?acc=hsa-mir-214 (accessed on Jan. 8, 2019).

An miRNA mimic intends a small, double-stranded RNA molecules designed to mimic endogenous mature miRNA molecules when introduced into cells. miR-214 mimics are commercially available from Sigma Aldrich see, sigmaaldrich/com/catalog/product/sigma/hmi0379?lang=en&region=US.

When a marker is used as a basis for selecting a patient for a treatment described herein, the marker is measured before and/or during treatment, and the values obtained are used by a clinician in assessing any of the following: (a) probable or likely suitability of an individual to initially receive treatment(s); (b) probable or likely unsuitability of an individual to initially receive treatment(s); (c) responsiveness to treatment; (d) probable or likely suitability of an individual to continue to receive treatment(s); (e) probable or likely unsuitability of an individual to continue to receive treatment(s); (f) adjusting dosage; (g) predicting likelihood of clinical benefits; or (h) toxicity. As would be well understood by one in the art, measurement of the genetic marker or polymorphism in a clinical setting is a clear indication that this parameter was used as a basis for initiating, continuing, adjusting and/or ceasing administration of the treatments described herein.

The term “treating” as used herein is intended to encompass curing as well as ameliorating at least one symptom of the condition or disease.

“An effective amount” intends to indicated the amount of a composition, compound or agent (exosomes) administered or delivered to the subject that is most likely to result in the desired response to treatment. The amount is empirically determined by the patient's clinical parameters including, but not limited to the stage of disease, age, gender and histology.

The term “blood” refers to blood which includes all components of blood circulating in a subject including, but not limited to, red blood cells, white blood cells, plasma, clotting factors, small proteins, platelets and/or cryoprecipitate. This is typically the type of blood which is donated when a human patient gives blood.

A “composition” is intended to mean a combination of active exosome or population of exosomes and another compound or composition, inert (e.g., a detectable label or saline) or active (e.g., a therapeutic compound or composition) alone or in combination with a carrier which can in one embodiment be a simple carrier like saline or pharmaceutically acceptable or a solid support as defined below.

A “pharmaceutical composition” is intended to include the combination of an active exosome or population of exosomes with a carrier, inert or active such as a solid support, making the composition suitable for diagnostic or therapeutic use in vitro, in vivo or ex vivo.

As used herein, the term “pharmaceutically acceptable carrier” encompasses any of the standard pharmaceutical carriers, such as a phosphate buffered saline solution, water, and emulsions, such as an oil/water or water/oil emulsion, and various types of wetting agents. The compositions also can include stabilizers and preservatives. For examples of carriers, stabilizers and adjuvants, see Martin (1975) Remington's Pharm. Sci., 15th Ed. (Mack Publ. Co., Easton).

A “subject,” “individual” or “patient” is used interchangeably herein, and refers to a vertebrate, preferably a mammal, more preferably a human. Mammals include, but are not limited to, murines, rats, rabbits, simians, bovines, ovines, porcines, canines, felines, farm animals, sport animals, pets, equines, and primates, particularly humans.

“Administration” can be effected in one dose, continuously or intermittently throughout the course of treatment. Methods of determining the most effective means and dosage of administration are known to those of skill in the art and will vary with the composition used for therapy, the purpose of the therapy, the target cell being treated, the disease being treated and the subject being treated. Single or multiple administrations can be carried out with the dose level and pattern being selected by the treating physician. Suitable dosage formulations and methods of administering the agents are known in the art. Route of administration can also be determined and method of determining the most effective route of administration are known to those of skill in the art and will vary with the composition used for treatment, the purpose of the treatment, the health condition or disease stage of the subject being treated, and target cell or tissue. Non-limiting examples of route of administration include oral administration, nasal administration, inhalation, injection, and topical application.

An agent of the present disclosure can be administered for therapy by any suitable route of administration. It will also be appreciated that the preferred route will vary with the condition and age of the recipient, and the disease being treated.

Diagnostic Methods

Provided herein are methods of determining whether a subject is suffering from endometriosis or an associated disorder, comprising, or alternatively consisting essentially of, or yet further consisting of, determining the expression level of miR-214 in a sample enriched from a subject, wherein reduced expression of the miR-214 as compared to a control is indicative of the subject suffering from endometriosis or an associated disorder. Further provided are methods of determining whether a subject suffering from endometriosis or an associated disorder is more or less likely to be responsive to a therapy to treat endometriosis or an associated disorder, comprising, or alternatively consisting essentially of, or yet further consisting of, determining the expression level of miR-214 in a sample enriched from the subject, wherein reduced expression of the miR-214 as compared to a control indicates the subject is more likely to be responsive to therapy and/or wherein increased expression of the miR-214 as compared to a control indicates the subject is less likely to be responsive to the therapy.

In one aspect, the expression level of the miR-214 is determined in a sample comprising an exosome enriched from a tissue or a body fluid of the subject. Non-limiting examples of such include, for example, blood, urine, saliva, breast milk, lymphatic fluid, serum and/or plasma. The enriched exosome sample can comprise whole exosomes or an exosome lysate. In a further aspect, the sample comprises endometrial stromal cells enriched from a subject.

In the methods of this disclosure, the reduced expression of the miR-214 is selected from: at least 1.5 fold, at least 2 fold, at least 2.5 fold, at least 3 fold, at least 3.5 fold, at least 4 fold, at least 4.5 fold, at least 5 fold, at least 5.5 fold, at least 6 fold, at least 6.5 fold, at least 7 fold, at least 7.5 fold, at least 8 fold, or at least 8.5 fold, reduced expression as compared to a control is indicative of the subject being more likely to be responsive to the therapy.

In a further aspect, the subjects identified to be responsive to the therapy are administered an effective amount of one or more of miR-214 or an equivalent thereof, miR-214-enriched exosome or an enriched equivalent miR-214 exosome, or miR-214 mimic, to the subject.

The methods are useful in the diagnosis and monitoring of a subject, e.g., a mammal, an animal, or yet further a human patient. For the purpose of illustration only, a mammal includes but is not limited to a human, a simian, a murine, a rat, a bovine, a canine, a feline, an equine, a porcine or an ovine.

Collection of body fluid samples, e.g., saliva, blood, saliva, tissue breast milk, lymphatic fluid, serum or plasma, for exosome miR analysis, can be done with methods known in the art.

In some embodiments, the exosomes are enriched from the sample prior to determination of the miR profile. The exosomes can be purified from the fluid using the methods disclosed herein in art-recognized methods, such as by ultracentrifugation as described by Thery et al. (2006) “Isolation and characterization of exosomes from cell culture supernatants and biological fluids” Curr. Protoc. Cell Biol., Chapter 3, or as disclosed in Hong et al. (2014) PLoS One 9(8):e103310, doe:10,1371 and Jayachandran et al. (2012) J. Immun. Methods, 375:207-214. Commercial kits also are available, e.g., PureExo (101BIO, Palo Alto, Calif., for serum and plasma), Exo MIR Plus (Bioo Scientific, Austin, Tex., USA), ExoQuick (SBI, Mountain View, Calif., USA, for tissue culture) and Exo-Spin Kit (Cell Guidance Systems, Carlsbad, Calif., USA). As apparent to the skilled artisan, the enrichment method will depend on the size and composition of the exosome to be enriched. As an example, ultracentrifugation can be used but for larger microvesicles, and the speed shall not exceed about 70,000 g or alternatively about 60,000 g. Alternatively, ultracentrifugation is used for smaller exosomes, but being much smaller, speeds of 90,000 g or alternatively of 100,000 g or more are needed. In some embodiments, the exosomes are enriched by ultracentrifugation. In some embodiments, the exosomes are enriched from a biological sample using an exosome surface marker. In some embodiments, the exosomes are retained on a capture surface sufficient to retain the exosome fraction on or in the capture surface. In some embodiments, the capture surface is positively charged. In some embodiments, the capture surface is a membrane comprising regenerated cellulose or quaternary ammonium. In some embodiments, the exosomes are enriched using an exosome-specific antibody. In some embodiments, the antibody specifically binds to Rab 5b, HSPA8, CD9, GAPDH, ACTB, CD63, CD81, ANXA2, ENO1, HSP9OAA1, EEF1A1, PKM2, YWHAE, SDCBP, PDCD61P, ALB, YWHAZ, EEF2, ACTG1, LDHA, HSP90AB1, ALDOA, MSN, ANXA5, PGK1, and CFL1. In some embodiments, the exosomes are enriched using size exclusion chromatography, filtration or immunosorbent capture.

In one aspect, the exosomes have an average diameter from about 10 to about 250 nm, or alternatively from about 10 to about 200 nm, or alternatively from about 10 to about 175 nm, or alternatively from about 25 to 175 nm, or alternatively from about 40 to about 250 nm, or alternatively from about 40 to about 200 nm, or alternatively from about 50 to 250 nm, or alternatively from about 50 to 200 nm, or alternatively from about 50-150 nm in average diameter. In another aspect, the term exosome also includes microvesicles that range from 100 nm to 1000 nm in approximate diameter.

In some embodiments, the analyzed sample of exosomes comprises whole exosomes or an exosome lysate.

Measurement of expression level or activity level can be accomplished by methods known in the art and briefly described herein, e.g., by PCR. The measurement can be compared to suitable controls, e.g., a prior measurement for that subject or a suitable internal control.

In regards to the disclosed methods, in some embodiments, said determining step comprises labeling the one or more miRNA with a detectable label, wherein in one aspect, the label is not a detectable polynucleotide.

In some embodiments, the determining step comprises capturing the one or more miRNA with one or more polynucleotide probe that selectively binds each of the one or more miRNA, or a nucleic acid array, or an in-situ hybridization.

In some embodiments, said determining step comprises using a real-time polymerase chain reaction or a nucleic acid array. In another aspect, the determining step comprises capturing the miR-214 with a polynucleotide probe that selectively binds miR-214.

The measurement of the above-noted miRNA markers can be combined with clinical parameters.

Compositions and Therapies

Further provided are compositions comprising enriched exosomes comprising miR-214 or a mimic thereof, alone or in combination with a carrier, such as a pharmaceutically acceptable carrier.

Further provided are methods of treating a subject suffering from endometriosis or an associated disorder by administering an effective amount of one or more of miR-214 or an equivalent thereof, miR-214-enriched exosome or an enriched exosome population comprising miR-214 equivalent, or miR-214 mimic to the subject, thereby treating endometriosis or an associated disorder. In one aspect, a sample enriched from the subject has been determined to have reduced level of miR-214 expression as compared to a control. Methods to determine expression level are described herein. One can monitor disease progression and/or treatment by periodically assaying for miR-214 expression in a sample isolated from the subject and/or by monitoring clinical symptoms of fibrosis and endometriosis in the patient. The disclosed methods can be combined with therapies known in the art and can be administered concurrently or sequentially as determined by the treating physician.

The compositions are useful in the diagnosis and treatment of a subject, e.g., a mammal, an animal, or yet further a human patient. For the purpose of illustration only, a mammal includes but is not limited to a human, a simian, a murine, a rat, a bovine, a canine, a feline, an equine, a porcine or an ovine.

miRNA, inhibitory RNA, antagomirs, and protectors can be prepared by any appropriate method, e.g., by isolation form natural products such as exosomes or recombinantly produced, for example, by a chemical synthetic method or a method using genetic recombination technique. When the production is carried out by a method using genetic recombination technique, miRNA can, for example, be produced through a transcription reaction with use of a DNA template and a RNA polymerase obtained by means of gene recombination. Examples of suitable RNA polymerase include a T7 RNA polymerase, a T3 RNA polymerase, and a SP6 RNA polymerase. They can be produced in a eukaryotic or prokaryotic cells, e.g., E. coli or other bacteria, yeast, mammalian, human, murine or simian for example.

In some aspects, the miRNAs are contained in or encoded by other nucleic acid molecules, and it is these nucleic acids that are isolated and purified for use in the described methods. The miRNAs can be contained within larger RNA molecules which, when processed, produce the miRNAs described herein. In another example, the miRNAs are encoded by nucleic acid molecules, which may be contained, for example, in vectors. Thus, also provided herein are vectors that contain nucleic acid that encodes the miRNAs.

In some instances, the miRNAs or nucleic acids encoding the miRNA and equivalents thereof are produced synthetically using well-known methods or are isolated from cells or tissues. Typically, the miRNAs or nucleic acid molecules containing or encoding the miRNAs are obtained using genetic engineering techniques to produce a recombinant nucleic acid molecule, which can then be isolated or purified by techniques well known to one of ordinary skill in the art. In these recombinant methods, nucleic acid encoding the miRNA is cloned into an appropriate expression vector. It is well within the skill of a skilled artisan to design DNA that encodes a miRNA provided herein.

Any suitable host/vector system can be used to express one or more of the miRNAs described herein. It is well with the skill of those in the art to select an appropriate system based on, for example, whether the miRNA or nucleic acid molecule encoding the miRNA or equivalent thereof is being isolated and purified for subsequent use, and/or whether the miRNA will be expressed in vivo following administration to a subject.

In particular examples, the miRNAs described herein (including precursor miRNAs) are encoded by vectors for expression of the miRNA or equivalent thereof in vivo following administration of the vector to a subject. The choice of vector, including the particular regulatory elements contained in the vector for expression of heterologous nucleic acid, can be influenced by the cell type to which the vector is being targeted, and such selection is well within the level of skill of the skilled artisan. For example, the nucleic acid encoding the miRNA or equivalent thereof can be under the control of a tissue- or cell-specific promoter, such that the miRNA is only expressed in that particular tissue or cell type. Tissue- or cell-specific promoters are well known in the art. Further provided for use in the methods are DNA polynucleotides encoding miR-214 and equivalents thereof.

In further examples, the nucleic acid encoding the miRNA and/or equivalent thereof is cloned into a viral vector, including, but not limited to, retroviral, adenoviral, lentiviral and adeno-associated viral vectors. Although viral vectors can be replication incompetent or replication competent, for subsequent use in therapeutic applications, typically replication incompetent vectors are selected.

Also provided are methods of producing an miR-214-enriched exosome comprising introducing a polynucleotide encoding miR-214, an miR-214 equivalent and/or an miR-214 mimic into a host cell or the miR-214, equivalent or miR-214 mimic, and culturing the cell under conditions that favor cell proliferation, and isolating the exosome enriched in the miR-214, equivalent or miR-214 mimic from the cells. In one aspect, the exosomes are enriched from a biological sample using an exosome surface marker. The host cell can be any appropriate prokaryotic or eukaryotic cell, e.g., a mammalian cell such as a human cell. In one aspect the cell is a stem cell or an endometrial stromal cell.

The expression level and activity of the miRNAs can be assessed using in vitro assays and animal models well known to those skilled in the art. The miRNAs also can be assessed in human clinical trials under appropriate supervision.

In one aspect, the non-diseased subject is one that is not suffering from endometriosis or an associated disorder. In another aspect, the exosomes have a microRNA (miR) profile comprising, or alternatively consisting essentially of, or yet further consisting of a typical or naturally occurring amount of miR-214, as compared to the miR profile of a subject that is suffering from endometriosis or an associated disorder.

Also provided is a purified or isolated population of exosomes isolated from a body fluid or tissue of a non-diseased subject, wherein the microRNA (miR) profile of the exosomes comprises, or alternatively consist essentially of, or yet further consist of, lack of reduced or down-regulation of miR-214 as compared to the miR profile of a subject that is suffering from endometriosis or an associated disorder.

The purified or isolated population of exosomes are isolated or purified from a body fluid selected from the group of tissue, stem cells, endometrial tissue, urine, lymphatic fluid, breast milk, saliva, blood, serum and/or plasma. The exosomes can be isolated from more than one source and combined or alternatively maintained as a tissue-specific sample.

This disclosure also provides pharmaceutical compositions comprising, or consisting essentially of, or yet further consisting of, purified or isolated exosomes and/or miRNA as described above. In one aspect, the pharmaceutical composition comprises, or alternatively consists essentially of, or yet further consists of, a pharmaceutically acceptable carrier and an effective amount of these exosomes isolated from a body fluid of a non-diseased subject. Non-limiting examples of carriers include phosphate buffered saline (PBS), saline or a biocompatible matrix material such as a collagen matrix. The compositions can optionally contain a protease inhibitor, glycerol and/or dimethyl sulfoxide (DMSO). They can be further formulated in liposomes or micelles, using methods known in the art.

For each of the above compositions, the fluid or tissue from which the exosomes are isolated or purified is selected from the group of tissue, stem cells, endometrial tissue, urine, breast milk, lymphatic fluid, saliva, blood, serum or plasma and can be present in a variety of concentrations.

The pharmaceutically acceptable carrier comprises one or more of a biocompatible matrix or a liquid carrier.

The pharmaceutical compositions of this disclosure can be formulated for freeze-drying or lyophilisation using methods known in the art, e.g., a cryoprotectant.

The pharmaceutical compositions are intended for in vitro and in vivo use. The compositions can comprise a concentration of exosomes and/or miRNA and/or inhibitory molecules (as measured by exosomal protein content (measured by Bicinchoninic protein assay (BCA), commercially available from Bio-Rad or Pierce Biotechnology, Inc., for example) from about 1 mg/ml to about 10 mg/ml, or alternatively from about 1 to about 8 mg/ml, or alternatively from 2 to about 8 mg/ml, or alternatively from 2 to about 5 mg/ml, or about 2 to 4 mg/ml, or alternatively from 3 mg/ml to 20 mg/ml When administered to the subject, an effective amount of the exosomes are administered to the subject, to cause at least about 75%, or alternatively at least about 80%, or alternatively at least about 85%, or alternatively at least about 90%, or alternatively at least about 95%, or alternatively at least about 99% effectiveness in the methods provided herein as compared to a control that does not receive the composition. Comparative effectiveness can be determined by suitable in vitro or in vivo methods as known in the art and briefly exemplified herein.

In one aspect, the compositions are pharmaceutical formulations for use in the therapeutic methods of this disclosure and for the treatment of the appropriate or relevant disease. While the examples are noted for the treatment of endometriosis, the principles can be applied to other disease conditions, including fibrotic disease in other organ or tissue as noted above. In the context of this disclosure when the exosomes and/or miRNA are isolated from a subject that is not suffering from endometriosis, the exosomes are useful for treating a subject having the disease.

In a further aspect, the disclosure provides a pharmaceutical composition comprising, or alternatively consisting essentially of, or yet further consisting of, the isolated or purified exosomes in a concentration such that composition comprises at least 75%, or alternatively at least 80%, or alternatively at least 85%, or alternatively at least 90%, or alternatively at least 95% or alternatively at least 97%, or alternatively at least 98%, or alternatively, at least 99% of exosomes (% noted as mg of exosomes and/or miRNA per mg of total proteins) in the total composition.

The compositions can be administered by oral, parenteral (e.g., intramuscular, intraperitoneal, intravenous, ICV, intracisternal injection or infusion, subcutaneous injection, or implant), by inhalation spray nasal, vaginal, rectal, sublingual, urethral (e.g., urethral suppository) or topical routes of administration (e.g., gel, ointment, cream, aerosol, etc.) and can be formulated, alone or together, in suitable dosage unit formulations containing conventional non-toxic pharmaceutically acceptable carriers, adjuvants, excipients, and vehicles appropriate for each route of administration. Non-limiting examples of carriers include phosphate buffered saline (PBS), saline or a biocompatible matrix material for topical or local administration. The compositions can optionally contain a protease inhibitor, glycerol and/or dimethyl sulfoxide (DMSO).

The pharmaceutical compositions can be conveniently presented in dosage unit form and can be prepared by any of the methods well known in the art of pharmacy. The pharmaceutical compositions can be, for example, prepared by uniformly and intimately bringing the active ingredient into association with a liquid carrier, a finely divided solid carrier or both, and then, if necessary, shaping the product into the desired formulation. In the pharmaceutical composition the active object compound is included in an amount sufficient to produce the desired therapeutic effect. For example, pharmaceutical compositions of the disclosure may take a form suitable for virtually any mode of administration, including, for example, topical, ocular, oral, buccal, systemic, nasal, injection, transdermal, rectal, and vaginal, or a form suitable for administration by inhalation or insufflation.

Systemic formulations include those designed for administration by injection (e.g., subcutaneous, intravenous, intramuscular, intrathecal, or intraperitoneal injection) as well as those designed for transdermal, transmucosal, oral, or pulmonary administration.

Useful injectable preparations include sterile suspensions, solutions, or emulsions of the active compound(s) in aqueous or oily vehicles. The compositions may also contain formulating agents, such as suspending, stabilizing, and/or dispersing agents. The formulations for injection can be presented in unit dosage form, e.g., in ampules or in multidose containers, and may contain added preservatives.

Alternatively, the injectable formulation can be provided in powder form for reconstitution with a suitable vehicle, including but not limited to sterile pyrogen free water, buffer, and dextrose solution, before use. To this end, the active compound(s) can be dried by any art-known technique, such as lyophilisation, and reconstituted prior to use.

For transmucosal administration, penetrants appropriate to the barrier to be permeated are used in the formulation. Such penetrants are known in the art.

For oral administration, the pharmaceutical compositions may take the form of, for example, lozenges, tablets, or capsules prepared by conventional means with pharmaceutically acceptable excipients such as binding agents (e.g., pregelatinised maize starch, polyvinylpyrrolidone, or hydroxypropyl methylcellulose); fillers (e.g., lactose, microcrystalline cellulose, or calcium hydrogen phosphate); lubricants (e.g., magnesium stearate, talc, or silica); disintegrants (e.g., potato starch or sodium starch glycolate); or wetting agents (e.g., sodium lauryl sulfate). The tablets can be coated by methods well known in the art with, for example, sugars, films, or enteric coatings. Additionally, the pharmaceutical compositions containing the 2,4-substituted pyrmidinediamine as active ingredient or prodrug thereof in a form suitable for oral use may also include, for example, troches, lozenges, aqueous, or oily suspensions, dispersible powders or granules, emulsions, hard or soft capsules, or syrups or elixirs.

Compositions intended for oral use can be prepared according to any method known to the art for the manufacture of pharmaceutical compositions, and such compositions may contain one or more agents selected from the group consisting of sweetening agents, flavoring agents, coloring agents, and preserving agents in order to provide pharmaceutically elegant and palatable preparations. Tablets contain the active ingredient (including drug and/or prodrug) in admixture with non-toxic pharmaceutically acceptable excipients which are suitable for the manufacture of tablets. These excipients can be for example, inert diluents, such as calcium carbonate, sodium carbonate, lactose, calcium phosphate or sodium phosphate; granulating and disintegrating agents (e.g., corn starch or alginic acid); binding agents (e.g., starch, gelatin, or acacia); and lubricating agents (e.g., magnesium stearate, stearic acid, or talc). The tablets can be left uncoated or they can be coated by known techniques to delay disintegration and absorption in the gastrointestinal tract and thereby provide a sustained action over a longer period. For example, a time delay material such as glyceryl monostearate or glyceryl distearate can be employed. They may also be coated by the techniques described in the U.S. Pat. Nos. 4,256,108; 4,166,452; and 4,265,874 to form osmotic therapeutic tablets for control release. The pharmaceutical compositions of the disclosure may also be in the form of oil-in-water emulsions.

Liquid preparations for oral administration may take the form of, for example, elixirs, solutions, syrups, or suspensions, or they can be presented as a dry product for constitution with water or other suitable vehicle before use. Such liquid preparations can be prepared by conventional means with pharmaceutically acceptable additives such as suspending agents (e.g., sorbitol syrup, cellulose derivatives, or hydrogenated edible fats); emulsifying agents (e.g., lecithin, or acacia); non-aqueous vehicles (e.g., almond oil, oily esters, ethyl alcohol, cremophore™, or fractionated vegetable oils); and preservatives (e.g., methyl or propyl-p-hydroxybenzoates or sorbic acid). The preparations may also contain buffer salts, preservatives, flavoring, coloring, and sweetening agents as appropriate.

Preparations for oral administration can be suitably formulated to give controlled release or sustained release of the active compound, as is well known. The sustained release formulations of this disclosure are preferably in the form of a compressed tablet comprising an intimate mixture of compound of the disclosure and a partially neutralized pH-dependent binder that controls the rate of compound dissolution in aqueous media across the range of pH in the stomach (typically approximately 2) and in the intestine (typically approximately about 5.5).

To provide for a sustained release of the exosomes, one or more pH-dependent binders can be chosen to control the dissolution profile of the sustained release formulation so that the formulation releases compound slowly and continuously as the formulation is passed through the stomach and gastrointestinal tract. Accordingly, the pH-dependent binders suitable for use in this disclosure are those which inhibit exosome breakdown and/or release of its contents during its residence in the stomach (where the pH is-below about 4.5), and which promotes the release of a therapeutic amount of the compound of the disclosure from the dosage form in the lower gastrointestinal tract (where the pH is generally greater than about 4.5). Many materials known in the pharmaceutical art as “enteric” binders and coating agents have a desired pH dissolution property. The examples include phthalic acid derivatives such as the phthalic acid derivatives of vinyl polymers and copolymers, hydroxyalkylcelluloses, alkylcelluloses, cellulose acetates, hydroxyalkylcellulose acetates, cellulose ethers, alkylcellulose acetates, and the partial esters thereof, and polymers and copolymers of lower alkyl acrylic acids and lower alkyl acrylates, and the partial esters thereof. One or more pH-dependent binders present in the sustained release formulation of the disclosure are in an amount ranging from about 1 to about 20 wt %, more preferably from about 5 to about 12 wt % and most preferably about 10 wt %.

One or more pH-independent binders may be in used in oral sustained release formulation of the disclosure. The pH-independent binders can be present in the formulation of this disclosure in an amount ranging from about 1 to about 10 wt %, and preferably in amount ranging from about 1 to about 3 wt % and most preferably about 2.0 wt %.

The sustained release formulation of the disclosure may also contain pharmaceutical excipients intimately admixed with the compound and the pH-dependent binder. Pharmaceutically acceptable excipients may include, for example, pH-independent binders or film-forming agents such as hydroxypropyl methylcellulose, hydroxypropyl cellulose, methylcellulose, polyvinylpyrrolidone, neutral poly(meth)acrylate esters, starch, gelatin, sugars, carboxymethylcellulose, and the like. Other useful pharmaceutical excipients include diluents such as lactose, mannitol, dry starch, microcrystalline cellulose and the like; surface active agents such as polyoxyethylene sorbitan esters, sorbitan esters and the like; and coloring agents and flavoring agents. Lubricants (such as talc and magnesium stearate) and other tableting aids can also be optionally present.

The pharmaceutical compositions can be in the form of a sterile injectable aqueous or oleaginous suspension. This suspension can be formulated according to the known art using those suitable dispersing or wetting agents and suspending agents which have been mentioned above. The sterile injectable preparation may also be a sterile injectable solution or suspension in a non-toxic parenterally-acceptable diluent or solvent. Among the acceptable vehicles and solvents that can be employed are water, Ringer's solution, and isotonic sodium chloride solution. The compositions may also be administered in the form of suppositories for rectal or urethral administration of the drug.

For topical use, creams, ointments, jellies, gels, solutions, suspensions, etc., containing the compounds of the disclosure, can be employed. In some embodiments, the compounds of the disclosure can be formulated for topical administration with polyethylene glycol (PEG). These formulations may optionally comprise additional pharmaceutically acceptable ingredients such as diluents, stabilizers, and/or adjuvants.

Included among the devices which can be used to administer compounds of the disclosure, are those well-known in the art, such as metered dose inhalers, liquid nebulizers, dry powder inhalers, sprayers, thermal vaporizers, and the like. Other suitable technology for administration of particular compounds of the disclosure, includes electrohydrodynamic aerosolizers. As those skilled in the art will recognize, the formulation of compounds, the quantity of the formulation delivered, and the duration of administration of a single dose depend on the type of inhalation device employed as well as other factors. For some aerosol delivery systems, such as nebulizers, the frequency of administration and length of time for which the system is activated will depend mainly on the concentration of compounds in the aerosol. For example, shorter periods of administration can be used at higher concentrations of compounds in the nebulizer solution. Devices such as metered dose inhalers can produce higher aerosol concentrations and can be operated for shorter periods to deliver the desired amount of compounds in some embodiments. Devices such as dry powder inhalers deliver active agent until a given charge of agent is expelled from the device. In this type of inhaler, the amount of compounds in a given quantity of the powder determines the dose delivered in a single administration.

Formulations of compounds of the disclosure for administration from a dry powder inhaler may typically include a finely divided dry powder containing compounds, but the powder can also include a bulking agent, buffer, carrier, excipient, another additive, or the like. Additives can be included in a dry powder formulation of compounds of the disclosure, for example, to dilute the powder as required for delivery from the particular powder inhaler, to facilitate processing of the formulation, to provide advantageous powder properties to the formulation, to facilitate dispersion of the powder from the inhalation device, to stabilize to the formulation (e.g., antioxidants or buffers), to provide taste to the formulation, or the like. Typical additives include mono-, di-, and polysaccharides; sugar alcohols and other polyols, such as, for example, lactose, glucose, raffinose, melezitose, lactitol, maltitol, trehalose, sucrose, mannitol, starch, or combinations thereof; surfactants, such as sorbitols, diphosphatidyl choline, or lecithin; and the like.

For prolonged delivery, the exosome compositions can be formulated as a depot preparation for administration by implantation or intramuscular injection. The active ingredient can be formulated with suitable polymeric or hydrophobic materials (e.g., as an emulsion in an acceptable oil) or ion exchange resins, or as sparingly soluble derivatives (e.g., as a sparingly soluble salt). Alternatively, transdermal delivery systems manufactured as an adhesive disc or patch which slowly releases the active compound(s) for percutaneous absorption can be used. To this end, permeation enhancers can be used to facilitate transdermal penetration of the active compound(s). Suitable transdermal patches are described in, for example, U.S. Pat. Nos. 5,407,713; 5,352,456; 5,332,213; 5,336,168; 5,290,561; 5,254,346; 5,164,189; 5,163,899; 5,088,977; 5,087,240; 5,008,110; and 4,921,475.

Alternatively, other pharmaceutical delivery systems can be employed. Liposomes and emulsions are well-known examples of delivery vehicles that can be used to deliver active compound(s) or prodrug(s). Certain organic solvents such as dimethylsulfoxide (DMSO) may also be employed, although usually at the cost of greater toxicity.

The pharmaceutical compositions may, if desired, be presented in a pack or dispenser device which may contain one or more unit dosage forms containing the active compound(s). The pack may, for example, comprise metal or plastic foil, such as a blister pack. The pack or dispenser device can be accompanied by instructions for administration.

The compositions will generally be used in an amount effective to achieve the intended result, for example, in an amount effective to treat or prevent the particular condition being treated. The compound(s) can be administered therapeutically to achieve therapeutic benefit or prophylactically to achieve prophylactic benefit. By therapeutic benefit is meant eradication or amelioration of the underlying disorder being treated and/or eradication or amelioration of one or more of the symptoms associated with the underlying disorder such that the patient reports an improvement in feeling or condition, notwithstanding that the patient may still be afflicted with the underlying disorder. Therapeutic benefit also includes halting or slowing the progression of the disease, regardless of whether improvement is realized.

The amount of compound administered will depend upon a variety of factors, including, for example, the particular condition being treated, the mode of administration, the severity of the condition being treated, the age and weight of the patient, the bioavailability of the particular active compound. Determination of an effective dosage is well within the capabilities of those skilled in the art. As known by those of skill in the art, the preferred dosage of compounds of the disclosure will also depend on the age, weight, general health, and severity of the condition of the individual being treated. Dosage may also need to be tailored to the sex of the individual and/or the lung capacity of the individual, where administered by inhalation. Dosage, and frequency of administration of the compositions will also depend on whether the compositions are formulated for treatment of acute episodes of a condition or for the prophylactic treatment of a disorder. A skilled practitioner will be able to determine the optimal dose for a particular individual.

For prophylactic administration, the compound can be administered to a patient at risk of developing one of the previously described conditions. For example, if it is unknown whether a patient is allergic to a particular drug, the compound can be administered prior to administration of the drug to avoid or ameliorate an allergic response to the drug. Alternatively, prophylactic administration can be applied to avoid the onset of symptoms in a patient diagnosed with the underlying disorder.

Effective dosages can be estimated initially from in vitro assays. For example, an initial dosage for use in animals can be formulated to achieve a therapeutic concentration and/or dosage of the exosome composition, as measured in an in vitro assay. Calculating dosages to achieve such effective dosages for other animal models or human patients is well within the capabilities of skilled artisans. For guidance, the reader is referred to Fingl & Woodbury, “General Principles,” In: Goodman and Gilman's The Pharmaceutical Basis of Therapeutics, Chapter 1, pp. 1-46, latest edition, Pergamagon Press, and the references cited therein.

Initial dosages can also be estimated from in vivo data, such as animal models. Animal models useful for testing the efficacy of compounds to treat or prevent the various diseases described above are well-known in the art. Ordinarily skilled artisans can routinely adapt such information to determine dosages suitable for human administration.

Dosage amounts of the miR-214 or equivalent thereof, miR-214 mimic or miR-214 or equivalent thereof-containing exosomes will typically be in the range of from about 0.0001 or 0.001 or 0.01 mg/kg/day to about 1000 mg/kg/day, but can be higher or lower, depending upon, among other factors, the activity of the composition, its bioavailability, the mode of administration, and various factors discussed above. Dosage amount and interval can be adjusted individually to provide local and/or systemic concentration of the exosomes that are sufficient to maintain therapeutic or prophylactic effect. For example, the compositions can be administered once per week, several times per week (e.g., every other day), once per day, or multiple times per day, depending upon, among other things, the mode of administration, the specific indication being treated, and the judgment of the prescribing physician. Skilled artisans will be able to optimize effective local dosages without undue experimentation.

Preferably, the compositions will provide therapeutic or prophylactic benefit without causing substantial toxicity. Toxicity of the compositions can be determined using standard pharmaceutical procedures. The dose ratio between toxic and therapeutic (or prophylactic) effect is the therapeutic index. Compositions that exhibit high therapeutic indices are preferred.

Kits

Also provided are kits for administration of the compositions and carrying out the diagnostic methods comprising the composition that may include an appropriate dosage amount. Kits may further comprise suitable packaging and/or instructions for use of the compositions and/or diagnostic methods. Kits may also comprise a means for the delivery of the at least one compositions and a device such as an inhaler, spray dispenser (e.g., nasal spray), syringe for injection, or pressure pack for capsules, tables, suppositories, or other device as described herein.

In one aspect, a kit comprising one or more probes and/or primers to determine the expression profile of miR-214.

In a further aspect, the kit further comprises detectable labels that in one aspect are attached to the probes and/or primers, wherein in one aspect, the detectable label is not a polynucleotide.

Additionally, the kits can contain the composition and reagents to prepare a composition for administration. The composition can be in a dry or lyophilized form or in a solution, particularly a sterile solution. When the composition is in a dry form, the reagent may comprise a pharmaceutically acceptable diluent for preparing a liquid formulation. The kit may contain a device for administration or for dispensing the compositions, including, but not limited to, syringe, pipette, transdermal patch, or inhalant.

The kits may include other therapeutic compounds for use in conjunction with the compounds described herein and as such, the methods as disclosed herein can contain other appropriate therapeutic compounds or agents. These compounds can be provided in a separate form or mixed with the compositions of the present disclosure. The kits will include appropriate instructions for preparation and administration of the composition, side effects of the compositions, and any other relevant information. The instructions can be in any suitable format, including, but not limited to, printed matter, videotape, computer readable disk, or optical disc.

Kits may also be provided that contain sufficient dosages of the compounds or composition to provide effective treatment for an individual for an extended period, such as a week, 2 weeks, 3, weeks, 4 weeks, 6 weeks, or 8 weeks or more.

Equivalents

Thus, it should be understood that although the present disclosure has been specifically disclosed by preferred embodiments and optional features, modification, improvement and variation of the disclosures embodied therein herein disclosed may be resorted to by those skilled in the art, and that such modifications, improvements and variations are considered to be within the scope of this disclosure. The materials, methods, and examples provided here are representative of preferred embodiments, are exemplary, and are not intended as limitations on the scope of the disclosure.

The disclosure has been described broadly and generically herein. Each of the narrower species and subgeneric groupings falling within the generic disclosure also form part of the disclosure. This includes the generic description of the disclosure with a proviso or negative limitation removing any subject matter from the genus, regardless of whether or not the excised material is specifically recited herein.

In addition, where features or aspects of the disclosure are described in terms of Markush groups, those skilled in the art will recognize that the disclosure is also thereby described in terms of any individual member or subgroup of members of the Markush group.

All publications, patent applications, patents, and other references mentioned herein are expressly incorporated by reference in their entirety, to the same extent as if each were incorporated by reference individually. In case of conflict, the present specification, including definitions, will control.

1. A method of determining whether a subject is suffering from endometriosis or an associated disorder, comprising determining the expression level of miR-214 in a sample enriched from a subject, wherein reduced expression of the miR-214 as compared to a control is indicative of the subject suffering from endometriosis or an associated disorder.

2. A method of determining whether a subject suffering from endometriosis or an associated disorder is more or less likely to be responsive to a therapy to treat endometriosis or an associated disorder, comprising determining the expression level of miR-214 in a sample enriched from the subject, wherein reduced expression of the miR-214 as compared to a control indicates the subject is more likely to be responsive to therapy and/or wherein increased expression of the miR-214 as compared to a control indicates the subject is less likely to be responsive to the therapy.

3. The method of claim 1, wherein the expression level of the miR-214 is determined in a sample comprising an exosome enriched from a body fluid of the subject.

4. The method of claim 3, wherein the body fluid comprises one or more of blood, urine, saliva, breast milk, lymphatic fluid, serum and/or plasma.

5. The method of claim 3, wherein the sample of exosomes comprises whole exosomes or an exosome lysate.

6. The method of claim 1, wherein the sample comprises endometrial stromal cells enriched from a subject.

7. The method of claim 2, wherein the reduced expression of the miR-214 is selected from: at least 1.5 fold, at least 2 fold, at least 2.5 fold, at least 3 fold, at least 3.5 fold, at least 4 fold, at least 4.5 fold, at least 5 fold, at least 5.5 fold, at least 6 fold, at least 6.5 fold, at least 7 fold, at least 7.5 fold, at least 8 fold, or at least 8.5 fold, reduced expression as compared to a control is indicative of the subject being more likely to be responsive to the therapy.

8. The method of claim 2, wherein the therapy comprises administration of an effective amount of one or more of miR-214-enriched exosome, miR-214 or miR-214 mimic to the subject.

9. A method of treating a subject suffering from endometriosis or an associated disorder, comprising administering an effective amount of one or more of miR-214, miR-214-enriched exosome or miR-214 mimic to the subject, thereby treating endometriosis or an associated disorder.

10. The method of claim 9, wherein a sample enriched from the subject has been determined to have reduced level of miR-214 expression as compared to a control.

11. (canceled)

12. (canceled)

13. A method of producing an miR-214-enriched exosome comprising introducing an miR-214 or mimic into an cell , culturing the cell under conditions that favor cell proliferation, and isolating the exosome enriched in the miR-214 mimic from the cell.

14. The method of claim 13, wherein the miR-214 or miR-214 mimic is introduced into the cell by transfection or by electroporation.

15. (canceled)

16. The method of claim 3, wherein the exosomes are enriched from a biological sample using an exosome surface marker.

17. The method of claim 2, wherein the therapy is delivered via intraperitoneal injections.

18.-19. (canceled)

20. The method of claim 1, wherein said determining step comprises labeling the miR-214 with a detectable label, wherein in one aspect, the detectable label is not a detectable polynucleotide.

21. The method of claim 1, wherein said determining step comprises capturing the miR-214 with a polynucleotide probe that selectively binds miR-214.

22. The method claim 1, wherein said determining step comprises using a real-time polymerase chain reaction, or a nucleic acid array, or an in-situ hybridization.

23. The method of claim 1, further comprising the step of administering a therapeutic drug for endometriosis or an associated disorder.

24. A kit comprising one or more probes and/or primers to determine the expression profile of the miR-214.

25.-31. (canceled)

32. The kit of claim 24, further comprising an effective amount of therapy to treat endometriosis or an associated disorder, and optional instructions for use.


SEQUENCE LISTING (1 sequence)

SEQ ID NO: 1 - RNA, 110 nt, Homo sapiens
ggccuggcuggacagaguugucaugugucugccugucuacacuugcugugcagaacauccgcucaccuguacagcaggcacagacaggcagucacaugacaacccagccu